(19) United States
(12) Patent Application Publication
(10) Pub. No.: US 2002/0120234 A1
(43) Pub. Date: Aug. 29, 2002

(54) Suction occluder for blood vessels and other body lumens

(76) Inventor: Bobby Kong, Ann Arbor, MI (US)

Correspondence Address: John G. Posa, Gifford, Krass, Groh, Sprinkle,, Anderson & Citkowski, P.C., 280 N. Old Woodward Ave., Suite 400, Birmingham, MI, 48009-5394, US

(21) Appl. No.: 10/074,622

(22) Filed: Feb. 13, 2002

Related U.S. Application Data
(60) Provisional application No. 60/268,324, filed on Feb. 13, 2001.

(51) Int. Cl. A61M 29/00

(52) U.S. Cl. 604/103.07; 606/194

(57) ABSTRACT

A device, system and method are disclosed for occluding a body lumen such a blood vessel having an inner wall. A blocking element is provided having an outer periphery with one or more grooves, recesses or depressions. A tube or lumen is used to interconnect the vacuum source to the grooves, recesses or depressions, such that the suction of the vacuum source causes a water-tight seal to be established between the periphery of the element and the inner wall of the body lumen. To bring the outer periphery of the blocking element in closer proximity to the inner wall of the body lumen, the element may be inflatable with a liquid or gas through a separate tube or lumen. A system-level implementation would include a source of vacuum along with the blocking element having an outer periphery with one or more grooves, recesses or depressions. Regardless of embodiment, a monitor may be provided for ensuring that the level of suction is within a desirable range. The system may further include a source of inflation to expand the element within the lumen, in which case a monitor may also be used for ensuring that the level of pressurization is within a desirable range. The blocking element may be introduced into the lumen via a puncture hole, with the suction and/or inflation tubing preferably extending outwardly from the same puncture hole. Alternatively, depending upon vessel size, the element may be introduced with a catheter, in which case the suction line and inflation line (if used) would be operated from the proximal end of the catheter outside the body.

### REFERENCE TO RELATED APPLICATIONS

This application claims priority from U.S. provisional patent application Serial No. 60/268,324, filed Feb. 13, 2001, the entire contents of which is incorporated herein.

### FIELD OF THE INVENTION

This invention relates generally to body lumen occlusion and, in particular, to an occluder which uses suction to engage with the inner wall of a body lumen.

### BACKGROUND OF THE INVENTION

The selective occlusion of blood vessels is important in many therapeutic treatments, including the control of internal bleeding, the termination of blood supply to tumors, isolation of diseased body organs prior to removal, relief of blood pressure in a region of aneurysm, and others. While such procedures rely generally on the blockage of arteries, the selective occlusion of veins is also useful in certain procedures.

Different occluding devices have been developed for these purposes, including removable and permanent balloons, thrombosing (clogging) coils, sclerosing (hardening) drugs, and fast-acting embolization glue (often used before surgery). Most of these devices are deployed through the use of endovascular catheters.

Each type of device has its advantages and disadvantages in terms of effectiveness, placement accuracy, and so forth. Embolization glues solidify on a vessel wall as a function of exposure to electrolytes in the blood. As such, accurate placement is a function of cure rate. Thrombosis-producing particles may alternatively be used, these being formed of various materials such as polyvinyl alcohol, silicone polymer, protein particles, glass beads, latex beads, or silk suture. The blockage may be temporary or permanent, depending on whether and to what degree the particle is broken down in the body, resulting in recanalization of a blood vessel after occlusion.

Mechanical endoluminal techniques include the use of detachable balloons, embolic and vaso-occlusion coils, and the like, to physically block the vessel lumen. Detachable balloons are typically advanced to the vessel site at the end of a catheter and inflated with a suitable fluid, such as saline, x-ray contrast or a polymerizable resin, and released from the end of the catheter. The method of detachment is usually based upon friction against the vessel wall, leading to resistance to withdrawal as the catheter is pulled out. Particularly with larger vessels, coaxial detachment may be used, which involves translation of a larger catheter over a smaller catheter containing the balloon. This permits the inner catheter to be removed from the balloon while the balloon maintains its position.

Balloon occlusion devices can sometimes deflate or rupture, however, leading to unpredictable circumstances and, in some cases, complications. A more recent alternative, particularly for smaller vessels, is the endovascular “coil,” which is typically a stainless steel wire wound such that its outer diameter matches the inner diameter of an angiographic catheter. These embolic or vaso-occlusion coils are typically introduced through the catheter in a stretched, linear form, and assume a relaxed, helical shape when released into a vessel. This produces an obstacle in the blood vessel, resulting in clotting and eventual blockage. Further development resulted in the addition of fibers of cotton or other material within the coil, promoting more rapid thrombosis.

One of the limitations of these coils is that recanalization of the occlusion site can occur when the initial blood clot is broken down by the body's natural anticoagulant mechanism (i.e., resorption of the clot). In addition, once the embolic coils are released by the introducer catheter, they are no longer under control and they frequently migrate from the point of initial implantation. To completely arrest the flow of blood in a vessel and to inhibit recanalization, current methods of coil embolization typically require the use of several embolic coils used in a “nesting technique” at the target site in the blood vessel.

It has been found, however, that the use of several coils does not always prevent recanalization of the blood vessel, particularly in larger, high flow vessels. Moreover, it often takes a relatively long time for the blood vessel to completely occlude. Therefore, the embolic coils may often migrate into a non-target site prior to vessel occlusion, particularly in larger or high flow vessels. Multiple coils are also more expensive than a single coil and they require more time to position within the vessel, thereby further increasing the cost of the procedure and prolonging the patient's exposure to the fluoroscope.

Regardless of the approach, success often rests on the ability of the device to be precisely placed, and its ability to adhere to the vessel wall. To enhance targeting and efficacy some techniques involve the use of mechanical clamping. For example, in open surgical and endoscopic procedures, the body vessel may be externally clamped and radio frequency energy applied. While the external procedures can be very effective, it requires external access to the lumen and is unsuitable for endoluminal techniques.

U.S. Pat. No. 6,042,563 describes a method and apparatus for occluding a blood vessel which uses both external clamping and internal inflation. A cannula adapted for insertion through a wall of a blood vessel is provided with an expandable member on a distal end which, when expanded, substantially fills a cross-sectional annular area of the lumen of the blood vessel. An external clamp is coupled to the cannula and aligned with the expandable member, such that when the clamp is engaged, it moves the annular region of the blood vessel into contact with the inflatable member, the inflatable member and clamp thereby working in cooperation to occlude the blood vessel.

Despite these advances, the need remains for a simple yet effective occlusion device, preferably deployed with minimal deformation to the blood vessel, thereby reducing the risk of trauma to the blood vessel and the creation of emboli. Such an intraluminal occlusion device should also remain securely placed in a selected position, thereby reducing risk of harm to the patient from emboli and undesirable blocking of perfusion of blood to the rest of the patient's body.

### SUMMARY OF THE INVENTION

This invention improves upon the existing art by providing a device, system and method for occluding a body lumen such a blood vessel having an inner wall. In the preferred embodiment, a blocking element is provided having an outer periphery with one or more grooves, recesses or depressions. A tube or lumen is used to interconnect the vacuum source to the grooves, recesses or depressions, such that the suction of the vacuum source causes a water-tight seal to be established between the periphery of the element and the inner wall of the body lumen.

The blocking element may be any appropriate shape such as a balloon, though a disc or membrane is used in the preferred embodiment. To bring the outer periphery of the blocking element in closer proximity to the inner wall of the body lumen, the element may be inflatable with a liquid or gas through a separate tube or lumen.

A system for occluding a body lumen according to the invention would include a source of vacuum along with the blocking element having an outer periphery with one or more grooves, recesses or depressions. Regardless of embodiment, a monitor may be provided for ensuring that the level of suction is within a desirable range. The system may further include a source of inflation to expand the element within the lumen, in which case a monitor may also be used for ensuring that the level of pressurization is within a desirable range.

The blocking element may be introduced into the lumen via a puncture hole, with the suction and/or inflation tubing preferably extending outwardly from the same puncture hole. Alternatively, depending upon vessel size, the element may be introduced with a catheter, in which case the suction line and inflation line (if used) would be operated from the proximal end of the catheter outside the body.

### BRIEF DESCRIPTION OF THE DRAWINGS

FIG. 1 is a drawing which shows a preferred embodiment of the invention; and

FIG. 2 is a drawing which shows an alternative embodiment of the invention.

### DETAILED DESCRIPTION OF THE INVENTION

This invention relates to device to at least temporarily occlude vessels in human and animals, including vessels associated with the cardiovascular system. However, the invention is not limited in this regard, it may be used to include any vessel, duct, or passageway having an inner wall.

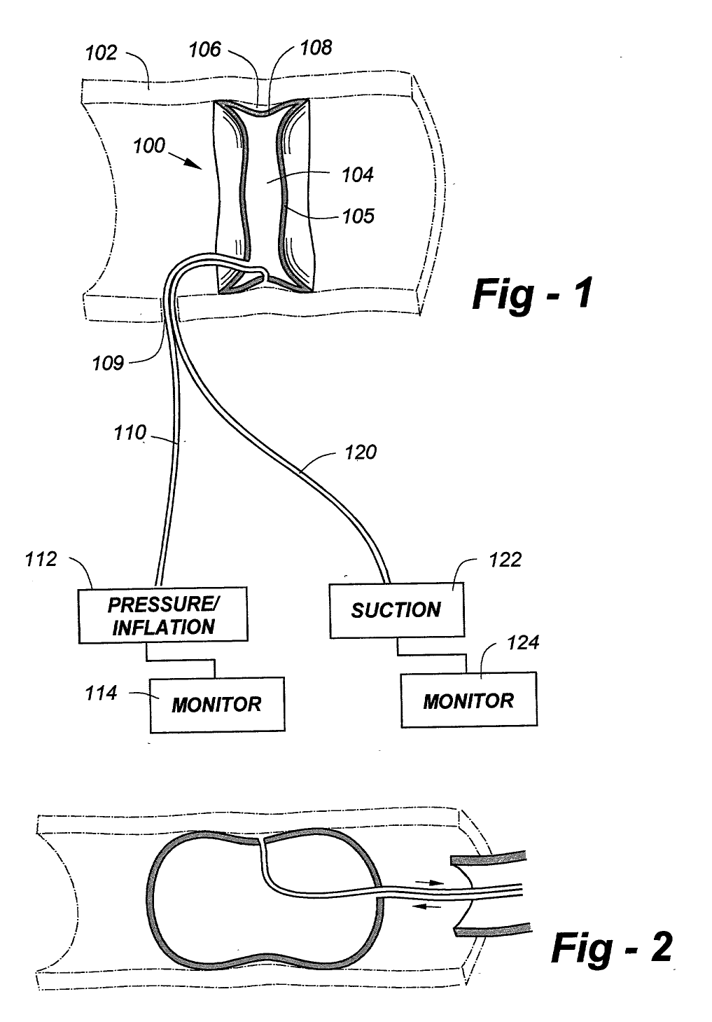

FIG. 1 is a drawing which shows a preferred embodiment of the invention, including a vessel-blocking element depicted generally at 100 having an outer periphery with one or more grooves, recesses or depressions. A vacuum or suction source 122 is coupled to these grooves, recesses or depressions through line 120, allowing the blocking element to maintain a “water-tight” seal to the inner wall of the lumen 102.

The grooves, recesses or depressions may take the form of a series of suction areas, or, in the preferred embodiment, comprises a continuous airtight channel 106 defined by wall 108 located peripherally around the blocking element 100. The blocking element 100 itself may take the form of a non-inflatable disc, membrane, or other shape though, in the preferred embodiment, the element 100 includes an inflatable cavity 104 defined by walls 105. This allows inflation from source 112 through line 110 while the suction/vacuum is applied, thereby maintaining a high-integrity, water-tight seal.

The pressure/inflation and suction sources 112, 122 may be disposed in the same piece of equipment, with monitors 114, 124 preferably being used to ensure that the pressurization and vacuum levels are within appropriate ranges indicative of proper placement and deployment. A gas or a liquid may be used for pressurization.

The element 100 may be introduced into the vessel via a puncture hole 109, with the suction and/or inflation tubing preferably extending outwardly from the same puncture hole. Alternatively, depending upon vessel size, the element 100 may be introduced with a catheter, in which case the suction line and inflation line (if used) would be operated from the proximal end of the catheter outside the body. Particularly in this embodiment, it may be advantageous to shape the element 100 more like a balloon, which may be self-expanding or inflated from an external source (see FIG. 2).

I claim:

1. A device adapted for use with a vacuum source for occluding a body lumen having an inner wall, the device comprising:
a blocking element having an outer periphery with one or more grooves, recesses or depressions; and
a tube or lumen interconnecting the vacuum source to the grooves, recesses or depressions, such that the suction of the vacuum source causes a water-tight seal to be established between the periphery of the element and the inner wall of the body lumen.

2. The device of claim 1, wherein the vessel blocking element is shaped as a disc or membrane.

3. The device of claim 1, wherein the blocking element is inflatable with a liquid or gas to bring the outer periphery in close proximity to the inner wall of the lumen.

4. The device of claim 1, wherein the lumen forms part of a human cardiovascular system.

5. A device adapted for use with a vacuum source and an inflation source to occlude a body lumen having an inner wall, the device comprising:
an inflatable blocking element having an inner cavity an outer periphery with one or more grooves, recesses or depressions;
a first tube or lumen interconnecting the inflation source to the inner cavity of the blocking element; and
a second tube or lumen interconnecting the vacuum source to the grooves, recesses or depressions, such that pressurization of the cavity and suction to the grooves, recesses or depressions causes a water-tight seal to be established between the periphery of the device and the inner wall of the lumen.

6. The device of claim 5, wherein the blocking element is shaped as a disc or membrane.

7. The device of claim 5, wherein a liquid or a gas is used to inflate the element.

8. The device of claim 5, wherein the lumen forms part of a human cardiovascular system.

9. A system for occluding a body lumen having an inner wall, comprising:
a source of vacuum;
a blocking element having an outer periphery with one or more grooves, recesses or depressions;
a tube or lumen interconnecting the vacuum source to the grooves, recesses or depressions to achieve a water-tight seal between the periphery of the element and the inner wall of the lumen.

10. The system of claim 9, wherein the blocking element is shaped as a disc or membrane.

11. The system of claim 9, further including a monitor for ensuring that the level of suction is within a desirable range.

12. The system of claim 9, further including a source of inflation to expand the element within the lumen.

13. The system of claim 12, further including a monitor for ensuring that the level of pressurization is within a desirable range.

14. The system of claim 12, wherein a liquid or a gas is used to expand the element.

15. The system of claim 6, further including a catheter for positioning the element within the body lumen prior to step of achieving a water-tight seal.

16. The system of claim 9, wherein the lumen forms part of a human cardiovascular system.

17. A system for occluding a body lumen having an inner wall, comprising:
an inflation source;
a vacuum source;
an inflatable blocking element having inner cavity and an outer periphery with one or more grooves, recesses or depressions;
a first tube or lumen interconnecting the inflation source to the inner cavity of the blocking element; and
a second tube or lumen interconnecting the vacuum source to the grooves, recesses or depressions, such that pressurization of the cavity and suction to the grooves, recesses or depressions causes a water-tight seal to be established between the periphery of the device and the inner wall of the lumen.

18. The system of claim 17, wherein the blocking element is shaped as a disc or membrane.

19. The system of claim 17, further including a monitor for ensuring that the level of suction is within a desirable range.

20. The system of claim 17, further including a monitor for ensuring that the level of inflation is within a desirable range.

21. The system of claim 17, wherein a liquid or a gas is used to expand the element.

22. The system of claim 17, further including a catheter for positioning the element within the body lumen prior to inflation.
